(19) United States
(12) Patent Application Publication
(10) Pub. No.: US 2019/0194600 A1
(43) Pub. Date: Jun. 27, 2019

(54) NOVEL CULTIVATION SYSTEM FOR THE EFFICIENT PRODUCTION OF MICROORGANISMS

(71) Applicant: LOCUS IP COMPANY, LLC, SOLON, OH (US)

(72) Inventors: KEN ALIBEK, SOLON, OH (US); SEAN FARMER, NORTH MIAMI BEACH, FL (US); PAUL S. ZORNER, ENCINITAS, CA (US); SHARMISTHA MAZUMDER, COPLEY, OH (US)

(21) Appl. No.: 16/331,430

(22) PCT Filed: Sep. 8, 2017
(86) PCT No.: PCT/US2017/050661

§ 371 Date: Mar. 7, 2019

Related U.S. Application Data
(60) Provisional application No. 62/404,516, filed on Oct. 5, 2016.
(60) Provisional application No. 62/385,057, filed on Sep. 8, 2016.

(51) Int. Cl. C12N 1/20; C12N 1/14; C09K 8/582; C09K 8/52; C05F 11/08; A01N 63/00; C02F 3/34; E21B 43/16; A01M 17/00; A01M 21/00

(52) U.S. Cl. CPC C12N 1/20; C12N 1/14; C09K 8/582; C09K 8/52; C05F 11/08; C22B 3/18; C02F 3/34; E21B 43/16; A01M 17/00; A01M 21/00; A01N 63/00

(57) ABSTRACT

The invention generally relates to the cultivation and growth of bacterial, fungal and yeast cells on pilot plant and industrial scales and, more particularly, to the cultivation and growth of microbial cells in/on hydrophilic particles containing pre-seeded nutrient media within a matrix of hydrophobic sand.

### CROSS-REFERENCE TO A RELATED APPLICATION

This application claims the benefit of U.S. Provisional Application Ser. No. 62/404,516, filed Oct. 5, 2016; and U.S. Provisional Application Ser. No. 385,057, filed Sep. 8, 2016, both of which are incorporated herein by reference in their entirety.

### BACKGROUND OF INVENTION

Cultivation of microorganisms such as bacteria, yeast and fungi is important for the production of a wide variety of useful bio-preparations. Microorganisms play crucial roles in, for example, food industries, pharmaceuticals, agriculture, mining, environmental remediation, and waste management.

For example, the benefits of symbiotic relationships involving fungi have long been understood. It follows then, that there exists an enormous potential for the use of fungi in a broad range of industries. The agricultural industry is perhaps the most important of all commercial applications for fungal products. As one example, commercial products containing beneficial mycorrhizal fungi, are available for small-scale use. The restricting factor in commercialization of mycorrhizal products is cost per propagule density, where it is particularly expensive and unfeasible to apply fungal products to large scale agricultural operations with sufficient inoculum to see the benefits.

Two principle forms of cultivation of microorganisms exist: submerged cultivation and surface cultivation. Bacteria, yeasts and fungi can all be grown using either the surface or submerged cultivation methods. Both cultivation methods require a nutrient medium for the growth of the microorganisms. The nutrient medium, which can either be in a liquid or a solid form, typically includes a carbon source, a nitrogen source, salts and appropriate additional nutrients and microelements. The pH and oxygen levels are maintained at values suitable for a given microorganism.

In the case of submerged cultivation, microorganisms are submerged in a liquid medium such as alcohol, oil or a nutrient broth. All nutrient components for the growth of microorganisms are obtained through absorption from the surrounding liquid medium. Oxygen is provided using one or more of various methods of aeration. One disadvantage of this technique is that the amount of oxygen that can be dissolved in the medium is often a limiting factor.

The submerged cultivation method is capital, operation, and labor extensive. For example, this cultivation method requires an extensive investment in equipment necessary for large scale up-stream production. Furthermore, down-stream processing is required for concentration. purification and drying for storage and distribution.

A further significant drawback to the large-scale submerged cultivation method compared to the surface cultivation method is the risk of contamination by other microbes or activation of a bacteriophage. Once a culture is contaminated, or infected with a bacteriophage, the contamination or bacteriophage lysis can quickly spread throughout liquid media, resulting in the destruction of the entire batch.

For industrial production, further disadvantages of a submerged fermentation system include: 1) the need for continuous agitation of cultivated microorganisms and substrates; 2) use of a large amount of water; 3) the need for a continuous oxygen supply; 4) larger volume to fermentation mash; 5) production of a large volume of liquid waste; and 6) a high energy requirement.

But the submerged cultivation method is more scalable compared to surface cultivation and is thus employed currently in the great majority of pilot and industrial production of microorganisms and their metabolites.

In the case of surface cultivation methods, microorganisms grow on the surface of a culture medium such as agar or a liquid medium. The culture medium provides the necessary nutrients. Surface cultivation is highly effective in providing a sufficient amount of oxygen from the surrounding air as well as efficient removal of metabolites, such as by absorption into the medium. Extensive contamination of a surface cultivation system is rare due to the limited surface area of the growing culture.

Surface cultivation does, however, have disadvantages. For example, surface cultivation is not highly scalable for pilot and industrial production compared to the submerged cultivation method. A large scale surface process is extremely labor intensive and requires wide surface areas for cultivation in sophisticated incubators that must provide aseptic conditions.

Surface cultivation also includes techniques for growing microorganisms on solids in a packed bed, in a fluidized bed, in a tray, in foamed medium, and on semipermeable membranes.

Solid state fermentation (SSF) has also been found useful for cultivation of microorganisms. SSF is defined as growth of microorganisms on solid substrates in a defined gas phase, but in the absence, or near absence, of a free water phase. SSF has been used for the development of bioprocesses such as bioremediation and biodegradation of hazardous compounds, biological detoxification of agro-industrial residues, biopulping and production of value-added products such as biologically active secondary metabolites, including antibiotics, alkaloids, plant growth factors, enzymes, organic acids, biosurfactants, and aroma compounds.

For an industrial production, increased interest in SSF exists because of certain advantages compared to submerged fermentation; however, SSF systems have disadvantages including 1) it is difficult to maintain the system due to lack of in situ sterilization; 2) mixing is a very difficult process; and 3) the existing SSF systems are only sterile at the initial stage and not thereafter.

There exists a need for efficient cultivation methods for mass production of microorganisms and microbial metabolites that have industrial applicability.

### BRIEF SUMMARY

The subject invention provides methods and materials for efficient cultivation of microorganisms and production of microbial growth by-products. The subject invention also provides apparatuses for such cultivation and production.

Advantageously, the methods of the subject invention combine attributes of both submerged and surface cultivation techniques and, thus, provide a hybrid cultivation system that takes advantage of the beneficial features of both submerged and surface cultivation while reducing negative features inherent in each method.

In one embodiment, the hybrid cultivation system is a three-step process comprising a first step where microbial inoculum is produced. This first step can be carried out utilizing, for example, standard fermentation in a liquid nutrient for a period of time and under conditions to build up cell numbers for use as an inoculum. The second step comprises mixing hydrophilic particles with a hydrophobic material to form a matrix. This step is followed by a third step of contacting the liquid inoculum produced in step 1 with the hydrophilic-hydrophobic matrix substance produced in step 2.

In another embodiment, the microbial inoculum produced in the first step is introduced to a hydrophilic particulate substrate in a second step. The third step then comprises mixing the inoculum-coated hydrophilic particles with a hydrophobic material to form the growth matrix.

In one embodiment, the step of mixing the hydrophilic particles with the hydrophobic material to form a matrix may occur prior to the step of producing the microbial inoculum.

Advantageously, individual particles, having inoculum associated therewith, are stabilized within the growth matrix formed by the hydrophobic material, thereby providing a large number of micro-reactors for microbial growth.

Advantageously, particles contained in the matrix are provided with an aseptic environment. Furthermore, if some particles become contaminated, it does not result in the contamination of the entire batch. Additionally, the individual wetted hydrophilic particles are provided with an advantageous microenvironment with reduced effects of inhibitory metabolites and sufficient access to oxygen, leading to an advantageously high microbial concentration per unit of culture volume.

The microorganisms grown according to the subject invention can be, for example, bacteria, yeast, fungi and multicellular organisms. The subject invention is particularly suitable for the cultivation of microorganisms for agriculture, industry, bioleaching, bioremediation, and other areas where microbial products are being used.

In one embodiment, the subject invention further provides a composition comprising at least one type of microorganism and/or at least one microbial metabolite produced by the microorganism that has been grown using the hybrid cultivation system of the subject invention. The microorganisms in the composition may be in an active or inactive form. The composition may also be in a dried form or a fluid form.

The cultivation method of the subject invention can be performed either in a batch or continuous processes.

In one embodiment, the subject invention provides equipment for the cultivation of microorganisms utilizing the hybrid cultivation system of the current invention.

Advantageously, the method and equipment of the subject invention reduce the capital and labor costs of producing microorganisms and their metabolites on a large scale. Furthermore, the cultivation process, according to the subject invention, reduces or eliminates the need to concentrate organisms after completing cultivation. The subject invention provides a cultivation method that not only substantially increases the yield of microbial products per unit of nutrient medium but simplifies production and facilitates portability.

Advantageously, in certain embodiments, the systems of the subject invention harness the power of naturally-occurring local microorganisms and their metabolic by-products to nourish, invigorate, and protect crop ecosystems and the communities and environments in which these ecosystems exist. Enhancement of local microbial populations can be advantageous in other settings as well, including, but not limited to, environmental remediation (such as in the case of an oil spill), animal husbandry, aquaculture, forestry, pasture management, turf, horticultural ornamental production, waste disposal and treatment, mining, oil recovery, and human health, including in remote locations.

According to one specific embodiment of the invention, mycorrhizal fungi are grown in a myriad of hydrophilic particles (e.g., hydrophilic sand, perlite, vermiculite, and/or diatomaceous earth) that have associated therewith pre-inoculated growth media. These particles are suspended in a hydrophobic matrix, effectively isolated from one another. This creates an environment where a plurality of individual micro-reactors are concurrently cultivating the fungi. The porous nature of the growth matrix allows for easy, efficient aeration of the entire culture. Additionally, the need for an aseptic environment and, as follows, the risk of contamination, is essentially eliminated due to the isolation of individual micro-reactors. The hydrophobic material is not conducive to contaminating microorganism growth, and thus any small number of contaminated particles will be outnumbered by particles proliferating the fungus. The system thus combines the isolation and aeration benefits of solid media with the high final cellular density and inoculum ease of liquid broth cultivation.

### DETAILED DISCLOSURE

The subject invention provides materials and methods for the efficient cultivation of microorganisms and the production of microbial growth by-products. These by-products can include, for example, metabolites, polymers, biosurfactants, enzymes, carbon dioxide, organic acids, and solvents.

The subject invention also provides systems for such cultivation and production. The subject invention further provides cultivation processes that are suitable for cultivation of microorganisms and production of microbial metabolites on a desired scale. Advantageously, the subject invention can be used for effective cultivation of microorganisms to high densities, with a lower risk of contamination, compared to conventional methods.

The methods of the subject invention combine both submerged and surface cultivation principles. The methods thus provide a hybrid cultivation system that takes advantage of the beneficial features of both submerged and surface cultivation, while reducing the negative features inherent in each method.

The subject invention further provides materials and methods for the production of biomass (e.g., viable cellular material), extracellular metabolites (e.g., both small and large molecules), and/or intracellular components (e.g., enzymes and other proteins). The microbes and microbial growth by-products of the subject invention can also be used for the transformation of a substrate, such as an ore, wherein the transformed substrate is the product.

The subject invention further provides microbe-based products, as well as uses for these products to achieve beneficial results in many settings including, for example, improved bioremediation and mining; waste disposal and treatment; enhancing livestock and other animal health; and promoting plant health and productivity by applying one or more of the microbe-based products.

In specific embodiments, the systems of the subject invention provide science-based solutions that improve agricultural productivity by, for example, promoting crop vitality; enhancing crop yields; enhancing insect and disease resistance; controlling insects, nematodes, diseases and weeds; improving plant nutrition; improving the nutritional content of agricultural and forestry and pasture soils; and promoting improved and more efficient water use.

In one embodiment, the hybrid cultivation system is a three-step process comprising a first step where microbial inoculum is produced. This first step can be carried out utilizing, for example, fermentation in a liquid nutrient broth for a duration and under conditions that result in the production of suitable cell numbers to be used as a source of inoculum. The second step comprises mixing hydrophilic particles with a hydrophobic material to form a matrix. This step is followed by a third step of contacting the liquid inoculum produced in step 1 with the hydrophilic-hydrophobic matrix produced in step 2.

In another embodiment, the microbial inoculum produced in the first step is introduced to a hydrophilic particulate substrate in a second step. The third step then comprises mixing the inoculum-coated hydrophilic particles with a hydrophobic matrix-forming material to faun the growth matrix.

In another embodiment, the microbial inoculum produced in the first step is introduced to a hydrophobic material in a second step. The third step then comprises mixing the inoculum and hydrophobic material with hydrophilic particles to form the growth matrix.

In one embodiment, the method for cultivation of microorganisms according to the subject invention does not include the step of growing the inoculum. Thus, the inoculum could be obtained, for example, from a third party. In this case the method comprises the steps of:

1) mixing a hydrophobic material and hydrophilic particles to create a matrix;

2) contacting the matrix with a medium inoculated with a microorganism of interest thereby creating a matrix of micro-reactors;

3) growing said microorganism within said micro-reactors.

Alternatively, the inoculum may be mixed with the hydrophobic material and/or hydrophilic particles prior to the mixing of the hydrophilic particles of the hydrophobic material.

The mixing of solid substrates (e.g., hydrophobic material and hydrophilic particles) and microorganism inoculant may occur either inside or outside of a growth system, e.g., a fermentation vessel. flow the solid substrates and inoculant are mixed is not limited, so long as an essentially homogeneous mixture containing a matrix of microbe-inoculated micro-reactors is obtained.

In one embodiment, the subject invention further provides a method for producing microbial metabolites such as proteins, peptides, polyunsaturated fatty acids, biosurfactants and lipids. In one embodiment, the method for producing a microbial growth by-product according to the subject invention comprises the steps of:

1) mixing a hydrophobic material and hydrophilic particles to form a matrix;

2) contacting the matrix with a medium inoculated with a microorganism of interest, thereby creating a growth matrix;

3) growing the microorganism within the growth matrix;

4) harvesting a growth by-product produced by said microorganism.

The microbial growth by-products produced by microorganism of interest may be retained in the microorganisms or secreted into the medium.

In another embodiment, the method for producing microbial growth by-products may further comprise steps of concentrating and purifying the by-product of interest.

In one embodiment, the subject invention further provides a composition comprising at least one type of microorganism and/or at least one microbial growth by-product produced by said microorganism. The microorganisms in the composition may be in an active or inactive form. The composition may or may not comprise the growth matrix in which the microbes were grown. The composition may also be in a dried form or a liquid form.

In one embodiment, the composition is suitable for agriculture. For example, the composition can be used to treat soil, plants, and seeds. The composition may also be used as a pesticide.

In one embodiment, the subject invention further provides customizations to the materials and methods according to the local needs. For example, the method for cultivation of microorganisms may be used to grow those microorganisms located in the local soil or at a specific oil well or site of pollution. In specific embodiments, local soils may be used as the solid substrates in the cultivation method for providing a native growth environment. Advantageously, these microorganisms can be beneficial and more adaptable to local needs.

The cultivation method according to the subject invention not only substantially increases the yield of microbial products per unit of nutrient medium but also improves the simplicity of the production operation. Furthermore, the cultivation process can eliminate or reduce the need to concentrate microorganisms after finalizing fermentation.

Advantageously, in one embodiment, the subject invention augments fungal propagule per gram capability compared to conventional cultivation methods by at least a 3 log increase.

Advantageously, the method does not require complicated equipment or high energy consumption, and thus reduces the capital and labor costs of producing microorganisms and their metabolites on a large scale. The microorganisms of interest can be cultivated at small or large scale on site and utilized. even being still encapsulated in the hydrophobic matrix. Similarly, the microbial metabolites can also be produced at large quantities at the site of need.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The cells may be in a vegetative state or in spore form, or a mixture of both. The cells may be planktonic or in a biofilm form, or a mixture of both. The by-products of growth may be, for example, metabolites, cell membrane components, expressed proteins, and/or other cellular components. The cells may be intact or lysed. In preferred embodiments, the cells are in the vegetative state and are present, with broth in which they were grown, in the microbe-based composition. The cells may be present at, for example, a concentration of 1×10⁴, 1×10⁵, 1×10⁶, 1×10⁷, 1×10⁸, 1×10⁹, 1×10¹⁰, or 1×10¹¹ or more cells per milliliter of the composition

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, buffers, appropriate carriers, such as water, added nutrients to support further microbial growth, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” refers to removing some or all of the microbe-based composition from a growth vessel.

### Microorganisms

The microorganisms grown according to the subject invention can be, for example, bacteria, yeast, fungi or multicellular organisms.

In one embodiment, the microorganisms are bacteria, including gram-positive and gram-negative bacteria. These bacteria may be, but are not limited to, for example, Escherichia coli, Rhizobium (e.g., Rhizobium japonicum, Sinorhizobium meliloti, Sinorhizobium fredii, Rhizobium leguminosarum biovar trifolii, and Rhizobium etli), Bradyrhizobium (e.g., Bradyrhizobium japonicum, and B. parasponia), Bacillus (e.g., Bacillus subtilis, Bacillus firmus, Bacillus laterosporus, Bacillus megaterium, Bacillus amyloliquifaciens), Azobacter (e.g., Azobacter vinelandii, and Azobacter chroococcum), Arhrobacter (e.g. Agrobacterium radiobacter), Pseudomonas (e.g., Pseudomonas chlororaphis subsp. aureofaciens (Kluyver)), Azospirillium (e.g., Azospirillumbrasiliensis), Azomonas, Derxia, Beijerinckia, Nocardia, Klebsiella, Clavibacter (e.g., C. xyli subsp. xyli and C. xyli subsp. cynodontis), cyanobacteria, Pantoea (e.g., Pantoea agglomerans), Sphingomonas (e.g., Sphingomonas paucimobilis), Streptomyces (e.g., Streptomyces griseochromogenes, Streptomyces qriseus, Streptomyces cacaoi, Streptomyces aureus, and Streptomyces kasugaenis), Streptoverticillium (e.g., Streptoverticillium rimofaciens), Ralslonia (e.g., Ralslonia eulropha), Rhodospirillum (e.g., Rhodospirillum rubrum), Xanthomonas (e.g., Xanthomonas campestris), Erwinia (e.g., Erwinia carotovora), Clostridium (e.g., Clostridium bravidaciens, and Clostridium malacusomae) and combinations thereof.

In another embodiment, the microorganism is a yeast. A number of yeast species are suitable for production according to the current invention, including, but not limited to, Saccharomyces (e.g., Saccharomyces cerevisiae, Saccharomyces boulardii sequela and Saccharomyces torula), Debaromyces, Issalchenkia, Kluyveromyces (e.g., Kluyveromyces lactis, Kluyveromyces fragilis), Pichia spp (e.g., Pichia pastoris), and combinations thereof.

In one embodiment, the microorganism is a fungus, including, but not limited to, for example, Starmerella, Mycorrhiza (e.g., vesicular-arbuscular mycorrhizae (VAM), arbuscular mycorrhizae (AM)), Mortierella, Phycomyces, Blakeslea, Thraustochytrium, Penicillium, Phythium, Entomophthora, Aureobasidium pullulans, Fusarium venenalum, Aspergillus, Trichoderma (e.g., Trichoderma reesei, T. harzianum, T. viride and T. hamatum), Rhizopus spp, endophytic fungi (e.g., Piriformis indica) and combinations thereof.

In specific embodiments, the microorganisms are Mycorrhizal fungi such as Glomus spp. and Acaulospora spp. The microorganism can also be arbuscular mycorrhizal fungi (AMF). Advantageously, the subject invention facilitates the resource-efficient and cost effective introduction of mycorrhizal inoculants into the agricultural industry on a commercial scale.

### Hydrophilic Particles

The hydrophilic particle is preferably one that is easily wetted with an aqueous inoculum. Preferably, the inoculum completely coats and adheres to the particle.

The hydrophilic particle is any hydrophilic substrate or material, such as, for example, vermiculite, perlite, hydrophilic metal-containing ore, amorphous silica granular clay diatomaceous earth, or any substrate coated with one or more hydrophilic compounds.

These materials form loose, airy granular structures, preferably having a particle size of 0.00001-50 mm and a large surface area, more preferably, having a particle size of 0.0001-10 mm in diameter.

The hydrophilic particle preferably has a water contact angle of less than 90°, 80°, 70°, 60°, 50°, 40°, 30°, or even 20°.

In preferred embodiments, the particle is porous such that the inoculum penetrates into pores, crevices and other open spaces. The particle may have a porosity of, for example, 5%, 10%, 20%, 30%, 40%, 50%, 60%, or more.

### Hydrophobic Material

The hydrophobic material is any hydrophobic material, such as, for example, fumed silica, hydrophobic sand, hydrophobic silica sand, and any particle coated with a hydrophobic compound.

Hydrophobic sand, which is commercially available as “Magic Sand,” can be made from sand coated with a hydrophobic compound. The presence of this hydrophobic compound causes the grains of sand to adhere to one another and form cylinders (to minimize surface area) when exposed to water. When the composition is removed from water, it is dry and free flowing. Magic Sand is also known as Aqua Sand.

The properties of hydrophobic sand can be achieved with ordinary beach sand, which contains tiny particles of pure silica, and exposing it to, for example, vapors of trimethylsilanol (CH₃)₃SiOH, an organosilicon compound. This is a water-repellent or hydrophobic organosilicon molecule that seals cracks or pits in sand particles and prevents water from sticking to it. Upon exposure, the trimethylsilane compound bonds to the silica particles while forming water. The exteriors of the sand grains are thus coated with hydrophobic groups. Magic Sand appears silvery in water because hydrogen bonding between water molecules causes the water to form a bubble around the sand.

Other hydrophobic coatings can be used to create the hydrophobic material. In preferred embodiments, the hydrophobic material has a hydrophobicity of at least about 50%, 75%, 90%, 100% or more of the hydrophobicity of sand coated with trimethylsilanol (e.g., Magic Sand).

In specific embodiments, the hydrophobic material is hydrophobic sand, which can be coarse or fine.

“Sand” encompasses the following: a) rock fragment or detrital particle smaller than a granule and larger than a coarse silt grain, having a diameter in range of 1/16 to 2 mm being somewhat rounded by abrasion in the course of transport, and b) a loose aggregate, unlithified mineral or rock particles of sand size; an unconsolidated or moderately consolidated sedimentary deposit consisting essentially of medium-grained elastics. The material is most commonly composed of quartz, and when the term “sand” is used without qualification, a siliceous composition is implied; but the particles may be of any mineral composition or mixture of rock or mineral fragments, such as coral sand. Also, sand encompasses a mass of such material, especially on a beach, desert, or in a streambed.

“Coarse sand” encompasses the following: a) a geologic term for a sand particle having a diameter in the range of 0.5 to 1 mm, and a loose aggregate of sand consisting of coarse sand particles, and b) an engineering term for a sand particle having a diameter in the range of 2 mm (retained on U.S. standard sieve No. 10) to 4.76 mm (passing U.S. standard sieve No. 4).

“Fine sand” encompasses the following: a) a geologic term for a sand particle having a diameter in the range of 0.125 to 0.25 mm, and a loose aggregate of sand consisting of fine sand particles, b) an engineering term for a sand particle having a diameter in the range of 0.074 mm (retained on U.S. standard sieve No. 200) to 0.42 mm (passing U.S. standard sieve No. 40).

The terms are defined according to the U.S. Bureau of Mines Dictionary of Mining, Minerals, and Related Terms.

In a preferred embodiment, the sand used according to the subject invention is fine sand.

### Inoculation

The subject invention involves a step whereby a microbial inoculant is added to a growth matrix or to a component of the growth matrix.

The inoculating step is typically carried out first utilizing an incubation process that provides proper conditions for growth and development of sufficient quantities of microbes to serve as an inoculant. The incubation process may be carried out in any setting that is suitable for producing such microbes, such as a laboratory or an industrial setting. The incubation process may be carried out, for example, under aerobic conditions. In other embodiments, the incubation process may include anaerobic fermentation. The inoculum may be in, for example, a liquid or solid form.

In one embodiment, the incubation step is carried out at about 5° to about 100° C., preferably, about 20° to about 60° C., more preferably, about 25° to about 50° C. for a duration that is proper to build up cell numbers as a source of inoculum. This time may be, for example, 8 hours, 12 hours, 24 hours, 48 hours, 4 days, 7 days or more. In one embodiment, the inoculating step is carried out at a pH between 2 and 12, preferably, between 4 and 10, more preferably, between 6 and 8.

In other embodiments the inoculant is obtained from a third party.

In certain embodiments, a controlled mass flow of the growth matrix, or a component of the growth matrix (e.g., hydrophilic or hydrophobic substrates, or the combination of the two) passes a point of inoculation where it is inoculated. The solids may be moved by traveling vertically or horizontally. The inoculated growth matrix is preferably mixed to homogeneity.

In certain embodiments, an inoculation liquid is introduced to the growth matrix, or component thereof, by, for example, a stream, a spray, a mist and the like, or a combination thereof. Furthermore, the inoculation liquid may be applied uniformly and continuously, or applied in pulses with intervals. In certain embodiments, the inoculant may be provided at a concentration of at least 10² CFU/mL, 10³ CFU/mL, 10⁴ CFU/mL, 10⁵ CFU/mL, or more.

In one embodiment, the volume ratio of the growth matrix to the inoculant is in a range of 25:1 to 1:1, preferably 20:1 to 5:1, 15:1, to 10:1, or about 12:1.

The ratio between the hydrophilic particles and the hydrophobic material is at least 1:25, 1:20, 1:15, 1:10, 1:5, 1:4, 1:3, 1:2, 1:1, 2:1, 3:1, or 5:1.

In a further embodiment, the solid substances wetted by the liquid medium inoculated with the microorganism of interest account for at least 10%, 20%, 30%, 40%, 50%, 60%, 70%, 80%, and 90% of the total volume of the cultivation system.

### Formation of the Growth Matrix

In one embodiment, the growth matrix is created by efficient mixing of the hydrophilic particles and the hydrophobic material. In specific embodiments, the hydrophilic particles have been wetted with a nutrient medium pre-seeded with the microorganisms of interest (the inoculant). The individual particles are stabilized within the hydrophobic material forming a growth matrix and thereby providing a large number of micro-reactors.

Advantageously, each particle contained in the matrix is provided with an aseptic environment within the culture. Therefore, if some particles are contaminated, it will not result in the contamination of the entire batch. Additionally, the individual wetted hydrophilic particle is provided with an advantageous microenvironment with reduced effects of inhibitory metabolites and having sufficient access to oxygen, thereby leading to a substantial increase of microbial concentration per unit of culture.

### Cultivation and Growth Medium

In one embodiment, the culture medium used according to the subject invention, may contain supplemental nutrients for the microorganism. Typically, these include carbon sources, proteins or fats, nitrogen sources, trace elements, and/or growth factors (e.g., vitamins, pH regulators). It will be apparent to one of skill in the art that nutrient concentration, moisture content, pH, and the like may be modulated to optimize growth for a particular microbe.

In one embodiment, the method includes supplementing the cultivation with a nitrogen source. The nitrogen source can be, for example, in an inorganic form such as potassium nitrate, ammonium nitrate ammonium sulfate, ammonium phosphate, ammonia, urea, and ammonium chloride, or an organic form such as proteins, and amino acids. These nitrogen sources may be used independently or in a combination of two or more.

The method can further comprise supplementing the cultivation with a carbon source. The carbon source is typically a carbohydrate, such as glucose, sucrose, lactose, fructose, trehalose, mannose, mannitol, and maltose; organic acids such as acetic acid, fumaric acid, citric acid, propionic acid, malic acid, malonic acid, and pyruvic acid; alcohols such as ethanol, propanol, butanol, pentanol, hexanol, isobutanol, and glycerol; fats and oils such as soybean oil, rice bran oil, olive oil, corn oil, sesame oil, and linseed oil; etc. These carbon sources may be used independently or in a combination of two or more.

In one embodiment, growth factors and trace nutrients for microorganisms are included in the medium. Inorganic nutrients, including trace elements such as iron, zinc, copper, manganese, molybdenum and cobalt may also be included in the medium.

In one embodiment, inorganic salts may also be included. Inorganic salts can be, for example, potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium carbonate. These inorganic salts may be used independently or in a combination of two or more.

Advantageously, the method provides easy oxygenation of the growing culture with, for example, slow motion of air to remove low-oxygen containing air and introduction of oxygenated air. The oxygenated air may be ambient air supplemented periodically, such as daily.

In some embodiments, the method for cultivation may further comprise adding additional acids and/or antimicrobials in the liquid medium before, and/or during the cultivation process. Antimicrobial agents or antibiotics are used for preventing the culture from contaminations. Additionally, antifoaming agents may also be added to prevent the formation and/or accumulation of foam when gas is produced during cultivation and fermentation.

In one embodiment, the method for cultivation of microorganisms is carried out at about 5° to about 100° C., preferably, 15° to 60° C., more preferably, 25 to 50° C. In a further embodiment, the cultivation may be carried out continuously at a constant temperature. In another embodiment, the cultivation may be subject to changing temperatures.

In one embodiment, the moisture level of the mixture should be suitable for the microorganism of interest. In a further embodiment, the moisture level may range from 20% to 90%, preferably, from 30 to 80%, more preferably, from 40 to 60%.

In one embodiment, the pH of the mixture should be suitable for the microorganism of interest. Buffering salts, and pH regulators, such as carbonates and phosphates, may be used to stabilize pH near an optimum value. When metal ions are present in high concentrations, use of a chelating agent in the liquid medium may be necessary.

The microbes can be grown in planktonic faun or as biofilm. In the case of biofilm. the vessel may have within it a substrate upon which the microbes can be grown in a biofilm state. The system may also have, for example, the capacity to apply stimuli (such as shear stress) that encourages and/or improves the biofilm growth characteristics.

### Growth Vessels

The microbe growth vessel used according to the subject invention can be any fermenter or cultivation reactor for industrial use. The cultivation process is carried out in a vessel that may be, for example, conical or tubular. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

Preferably, each growth vessel has its own controls and measuring systems for at least temperature and pH. In addition to monitoring and controlling temperature and pH, each vessel may also have the capability for monitoring and controlling, for example, dissolved oxygen, agitation, foaming, purity of microbial cultures, production of desired metabolites and the like.

In one embodiment, a single type of microbe is grown in a vessel. In alternative embodiments, multiple microbes, which can be grown together without deleterious effects on growth or the resulting product, can be grown in a single vessel. There may be, for example, 2 to 3 or more different microbes grown in a single vessel at the same time.

The growth vessel may be, for example, from 5 liters to 2,000 liters or more. Typically, the vessels will be from 10 to 1,500 liters, and preferably are from 100 to 1,000 liters, and more preferably from 250 to 750 liters, or from 400 to 600 liters.

These vessels may be, for example, made of glass, polymers, metals, metal alloys, and combinations thereof Prior to microbe growth, the vessel may be disinfected or sterilized.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of bacteria in a sample. The technique can also provide an index by which different environments or treatments can be compared.

In one embodiment, the fermentation vessel/reactor is a mobile or portable bioreactor that may be provided for on-site production of a microbiological product including a suitable amount of a desired strain of microorganism. Because the microbiological product (e.g., slurry or fluid) is generated on-site of the application, without resort to the bacterial stabilization, preservation, storage and transportation processes of conventional production, a much higher density of live microorganisms may be generated, thereby requiring a much smaller volume of the microorganism slurry for use in the on-site application. This allows for a scaled-down bioreactor (e.g., smaller fermentation tank, smaller supplies of starter material, nutrients, pH control agents, and de-foaming agent, etc.) that facilitates the mobility and portability of the system.

In one embodiment, the solid particles, fermentation medium, air, and equipment used in the method and cultivation process are sterilized. The cultivation equipment such as the reactor/vessel may be separated from, but connected to, a sterilizing unit, e.g., an autoclave. The cultivation equipment may also have a sterilizing unit that sterilizes in situ before starting the inoculation. The air can be sterilized by methods know in the art. For example, the ambient air can pass through at least one filter before supplemented into the vessel. In other embodiments, the medium may be pasteurized or optionally no heat at all added, where the use of low water activity and low pH may be exploited to control bacterial growth.

### Preparation of Microbe-Based Products

The microbe-based products of the subject invention include products comprising the microbes and/or microbial growth by-products and optionally, the hydrophobic material, the hydrophilic particles, and/or additional ingredients such as, for example, water, carriers, adjuvants, nutrients, viscousity modifiers, and other active agents.

One microbe-based product of the subject invention is simply the fermentation medium containing the microorganism and/or the microbial growth by-products produced by the microorganism and/or any residual nutrients. The product of fermentation may be used directly without extraction or purification. If desired, extraction and purification can be easily achieved using standard extraction methods or techniques described in the literature.

The microorganisms in the microbe-based product may be in an active or inactive form. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium resulting from the microbial growth can be removed from the growth vessel and transferred via, for example, piping for immediate use.

In other embodiments, the composition (microbes, medium, or microbes and medium) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, nutrients for plant growth, tracking agents, pesticides, herbicides, animal feed, food products and other ingredients specific for an intended use.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

The microbe-based products of the subject invention may be, for example, microbial inoculants, biopesticides, nutrient sources, remediation agents, health products, and/or biosurfactants.

In one embodiment, the fermentation products (e.g., microorganisms and/or metabolites) obtained after the cultivation process are typically of high commercial value. Those products containing microorganisms have enhanced nutrient content than those products deficient in the microorganisms. The microorganisms may be present in the cultivation system, the cultivation broth and/or cultivation biomass. The cultivation broth and/or bio mass may be dried (e.g., spray-dried), to produce the products of interest.

In one embodiment, the cultivation products may be prepared as a spray-dried biomass product. The biomass may be separated by known methods, such as centrifugation, filtration, separation, decanting, a combination of separation and decanting, ultrafiltration or microfiltration. The biomass cultivation products may be further treated to facilitate rumen bypass. The biomass product may be separated from the cultivation medium, spray-dried, and optionally treated to modulate rumen bypass, and added to feed as a nutritional source.

In one embodiment, the cultivation products may be used as an animal feed or as food supplement for humans. The cultivation products may be rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon. The peptides may contain at least one essential amino acid.

In other embodiments, the essential amino acids are encapsulated inside a subject modified microorganism used in a cultivation reaction. The essential amino acids are contained in heterologous polypeptides expressed by the microorganism. Where desired, the heterologous peptides are expressed and stored in the inclusion bodies in a suitable microorganism (e.g., fungi).

In one embodiment, the cultivation products have a high nutritional content. As a result, a higher percentage of the cultivation products may be used in a complete animal feed. In one embodiment, the feed composition comprises the modified cultivation products ranging from 15% of the feed to 100% of the feed.

It should be understood that the examples and embodiments described herein are for illustrative purposes only and that various modifications or changes in light thereof will be suggested to persons skilled in the art and are to be included within the spirit and purview of this application.

### EXAMPLE 1

### Bacterial Cultivation with Vermiculite as a Hydrophilic Substrate

Vermiculite, a hydrated magnesium aluminum silicate, is a cheap and commonly used product in gardening that is very porous.

The cultivation of Bacillus spp was carried out in a vessel where a small amount of liquid broth inoculum was introduced to the mixture of vermiculite and hydrophobic sand at a ratio of 1:12. This growth matrix was then mixed to homogeneity. The containing vessel was then incubated and received daily aeration treatments with ambient air.

Alternatively, a small amount of liquid broth inoculum (e.g., 10⁵ CFU/mL or 10⁴ CFU/mL) is mixed with the vermiculite in a 1/5 volumetric ratio. This mixture is blended well, leaving individual particles coated with inoculum. This mixture is further combined with a hydrophobic sand in a ratio ranging from 5:1 to 12:1 sand to inoculum, by volume, in an appropriate growth vessel containing aeration ports.

This can be further homogenized by agitation or mixing. The vessel is then incubated at appropriate temperatures and aerated at least three times per day.

To quantify cell density, a daily sample was taken and enumerated following serial dilution plating technique. Briefly, serial dilution of the sample is obtained. When fixed volumes of this dilution series are spread onto a solid growth medium and incubated, different numbers of colonies will be obtained. By noting the number of colonies, the volume of inoculant added, and the mass or volume of sample diluted, the number of microorganisms in the original sample can be calculated.

Under the conditions given in this example, in two days of cultivation, the cell concentration was grown from 10⁴ to at least 10⁹ CFU/mL.

### EXAMPLE 2

### Bacterial Cultivation with Perlite as a Hydrophilic Substrate

Perlite is a highly porous volcanic glass derived from obsidian. It is often used in insulation and gardening applications.

Following the same procedure as previously described for vermiculite, an optimal method was developed for the cultivation of Bacillus spp. utilizing perlite as a hydrophilic substrate. This involves adding liquid inoculum at a ratio of 1:12 to a perlite and hydrophobic sand mixture, which is homogenized and incubated.

Alternatively, a small amount of liquid broth inoculum (e.g., 10⁵ CFU/mL or 10⁴ CFU/mL) is mixed with the vermiculite in a 1/5 volumetric ratio. This mixture is blended well, leaving small individual saturated particles. This mixture is further combined with the hydrophobic sand matrix at a ratio from 5:1 to 12:1 sand to inoculum, by volume, in an appropriate growth vessel containing aeration ports.

This can be further homogenized by agitation or mixing. The vessel is then incubated at appropriate temperatures and aerated at least three times per day.

To quantify cell density, a daily sample was taken and enumerated following serial dilution plating technique.

Enumeration sampling showed that this method is also capable of reaching (from 10⁴) viable counts of at least 10⁹ CFU/mL within 2-3 days of inoculation.

### EXAMPLE 3

### Cultivation of Mycorrhiza Fungi with Diatomaceous Earth as a Hydrophilic Substrate

A method for the cultivation of Mycorrhiza utilizing hydrophobic sand and diatomaceous earth was also developed.

In this instance, a culture containing 10⁵ CFU/mL of the fungus is combined in a 1/5 ratio, by volume, to diatomaceous earth and blended to a fine particulate consistency. This is then further combined with the hydrophobic sand at a ratio from 5:1 to 12:1 sand to inoculum, by volume, preferably, 6:1 sand to inoculum ratio.

This mixture was allowed to incubate under optimal fungal growth conditions in a growth vessel, typically an appropriately sized glass bottle with aeration receptacles, and further shaken to homogeneity.

The sample was enumerated daily, yielding at least 10,000 propagules/gram (with initial concentration of 150 propagules/gram) within 2 weeks of inoculation.

### EXAMPLE 4

### Production of Microorganisms for Agriculture

The methods of the subject invention can be used to generate large amounts of concentrated cultures to be used for agricultural applications. Many examples of microbial application for agricultural purposes exist, including using Bacillus, Pseudomonas, and Mycorrhizae strains.

In certain embodiments, the method of the subject invention facilitates the production of large quantities of bacteria at the site in a close distance to application. The microbes can be grown on site and utilized still within the growth matrix. The microorganisms will integrate into the soil and microbes will be present in sufficient quantities to improve plant health and growth.

In one embodiment, the subject invention provides microbe-based compositions, as well as methods of using the compositions for promoting plant health, soil microbial diversity, plant nutrition, soil nutritive capacity, optimizing soil moisture status, soil aeration, soil water holding capacity and reducing the susceptibility of plants, to pests, diseases and weeds. This is achieved by improving a plant's natural defenses, the nutritive content, the microbial and toxilogical health of soils as well as by directly impacting plant pests, diseases or weeds. This plant-promotion effect occurs as a result of applying one or more of the microbe-based products of the subject invention to the plant and/or its environment.

In one embodiment, the compositions can be used to promote plant growth, yield and/or health. The composition may have activity against, for example, fungi and/or bacterial plant pathogens. The composition may also have activity against weeds. The composition may have activity against an insect pest of agriculture, turf, ornamentals, forestry and/or plant based production. The composition can be added to horticultural soil mixes; added at the time of planting of seed or transplants; or broadcast by hand or machine to agricultural fields, ponds, forests or any environment where it is desired to impact crops, animals and their pests.

### EXAMPLE 5

### Bioleaching

The present invention can be used in the process of bio-leaching. In this case, the process involves the cultivation of microorganisms capable of accumulating metals inside the cells. For example, Cupriavidus matallidurans can solubilize gold in ore to a soluble ionic form and convert into nanoparticles inside a cell.

Gold-containing ore can be used as micro-particles wetted with an appropriate nutrient medium for growing the bacterium in the matrix of hydrophobic sand.

Because this method does not require complicated equipment and high energy consumption, the installation for cultivation can be built at a site of ore.

### EXAMPLE 6

### Bioremediation

The present invention can be used to grow substantial quantities of bacteria and/or fungi that can be used to bio-remediate polluted soils and water.

Many microorganisms can be used for decontamination through bioremediation, including, for example, Pseudomonas, Arthrobacter and Bacillus strains. The microorganisms can be grown on site and released being still encapsulated in the hydrophobic matrix. The decontaminating microorganisms will be present in high numbers when introduced into the contaminated soil or water.

### EXAMPLE 7

### Oil Production

The subject invention provides microbe-based products, as well as their uses, in improved oil production. In certain embodiments, the subject invention provides materials and methods for improving oil production by treating drilling sites, including the wells and associated piping, with microorganisms and/or their by-products in order to enhance recovery of oil.

In some embodiments, the microbes can be salt-tolerant and/or surfactant over-producing microbes and by-products thereof. These by-products can include, for example, metabolites, polymers, biosurfactants, enzymes, carbon dioxide, organic acids, and solvents.

In preferred embodiments, such strains are characterized by enhanced biosurfactant production compared to wild type strains.

### EXAMPLE 8

### Pests of Structures

In one embodiment, the compositions of the subject invention have activity against pests of structures. These pests can be, for example, termites, ants or roaches. To control such pests, the composition can be applied, directly to the pests, or to the structure or the vicinity of the structure such that the pest will come into contact with the composition.

### EXAMPLE

### Pests of Animals

In another embodiment, the compositions of the subject invention can be used to control pests of animals, including humans. These pests can be, for example, mosquitoes, flies, nematodes, ticks, and fleas. To control such pests, the composition can be applied, directly to the pests, or to their environment such that the pest will come into contact with the composition. The microbes can be, for example, Bacillus thuriengensis. Control can include killing as well as reducing eating reproduction or other activity.

All patents, patent applications, provisional applications, and publications referred to or cited herein are incorporated by reference in their entirety, including all figures and tables, to the extent they are not inconsistent with the explicit teachings of this specification.

The description herein of any aspect or embodiment of the invention using terms such as “comprising,” “having,” “including,” or “containing” with reference to an element or elements is intended to provide support for a similar aspect or embodiment of the invention that “consists of,” “consists essentially of,” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method for cultivating microorganisms, wherein said method comprises the steps of:
a) mixing a hydrophobic material and hydrophilic particles to form a matrix;
b) contacting said matrix, said hydrophobic material, and/or said hydrophilic particles, with a medium inoculated with microorganisms, thereby creating a growth matrix; and
c) growing the microorganisms within said growth matrix.

2. The method according to claim 1, wherein the microorganisms are bacteria.

3-4. (canceled)

5. The method according to claim 1, wherein the microorganisms are fungi.

6. (canceled)

7. The method according to claim 1, wherein the hydrophobic material and hydrophilic particles are matrix-forming materials and are mixed with the medium inoculated with the microorganism at a ratio ranging from 20:1 to 5:1 of matrix-forming materials to medium.

8-9. (canceled)

10. The method, according to claim 1, wherein the hydrophilic particles have a water contact angle of 90° or less.

11. The method, according to claim 1, wherein the hydrophilic particles have a porosity of at least about 5%.

12. The method, according to claim 1, wherein the hydrophilic particles are selected from perlite, vermiculite, metal-containing ores, and diatomaceous earth.

13. The method according to claim 1, wherein the hydrophobic material is hydrophobic sand.

14. The method, according to claim 13, wherein the hydrophobic sand is coated with an organosilicon compound.

15-18. (canceled)

19. A composition comprising microorganisms, hydrophobic sand and hydrophilic particles wherein the hydrophilic particles are approximately 0.0001 to 10 mm in diameter and have a water contact angle of 90° or less, and wherein the microorganisms are present at a concentration of at least 10⁴ CFU/ml.

20. The composition, according to claim 19, wherein the hydrophilic particles have a porosity of at least about 5%.

21. The composition, according to claim 19, wherein the hydrophobic sand is coated with an organosilicon compound.

22-25. (canceled)

26. The composition, according to claim 19, wherein the microorganisms are bacteria.

27-28. (canceled)

29. The composition, according to claim 19, wherein the microorganisms are fungi.

30. The composition, according to claim 29, wherein the fungi are Mycorrhizal or Starmerella.

31. A method for enhancing the amount of oil recoverable from an oil-containing formation, wherein said method comprises applying a composition of claim 19 to the oil-containing formation.

32. (canceled)

33. A method for cleaning an oil well rod, tubing and/or casing, wherein said method comprises applying to the oil well rod, tubing and casing structures a composition of claim 19.

34. A method for improving plant growth, yield, and/or health, wherein said method comprises applying to the plant or its environment a composition of claim 19.

35-39. (canceled)

40. The method, according to claim 34, wherein the composition has activity against an insect pest of agriculture, turf, ornamentals, forestry and/or other plant based production.

41. The method, according to claim 34, wherein the composition has activity against weeds.

42. A method for controlling a pest of a structure wherein said method comprises applying to the structure, or the vicinity of the structure, or directly to the pest, a composition of claim 19.

43. A method for controlling a pest of animals wherein said method comprises contacting the pest with a composition of claim 19.

44-45. (canceled)

46. A method for bioleaching an ore wherein said method comprises administering to the ore a composition of claim 19.

47-48. (canceled)
